(19) United States
(12) Patent Application Publication
(10) Pub. No.: US 2018/0368889 A1
(43) Pub. Date: Dec. 27, 2018

(54) SPINAL FIXATION DEVICE

(71) Applicant: K2M, Inc., Leesburg, VA (US)

(72) Inventor: Chad Cole, St. George, UT (US)

(21) Appl. No.: 15/631,502

(22) Filed: Jun. 23, 2017

(51) Int. Cl. A61B 17/70; A61B 17/86

(52) U.S. Cl. CPC A61B 17/7032; A61B 90/03; A61B 17/8605; A61B 17/7001

(57) ABSTRACT

A spinal fixation device includes a housing assembly and a bone screw. The housing assembly includes an inner housing and an outer housing selectively slidable relative to each other between a locked position and an unlocked position. The bone screw includes a head portion selectively securable within the inner housing of the housing assembly, a neck portion, and an elongated body portion including a proximal region and a distal region. The distal region includes a threaded section having threads and an unthreaded section extending distally from the threaded section and terminating at a distal tip. The threaded section includes a cutting flute defined therein proximal to the unthreaded section.

### TECHNICAL FIELD

The present disclosure relates generally to a spinal fixation device, and more particularly, to devices, systems, and surgical methods for securing a spinal fixation device during orthopedic spine surgery.

### BACKGROUND

The spinal column is a complex system of bones and connective tissues that provide support for the human body and protection for the spinal cord and nerves. The adult spine includes an upper portion and a lower portion. The upper portion has twenty-four discrete bones, which are subdivided into three areas including seven cervical vertebrae, twelve thoracic vertebrae, and five lumbar vertebrae. The lower portion has the sacral and coccygeal bones. The cylindrical shaped bones, called vertebral bodies, progressively increase in size from the upper portion downwards to the lower portion.

An intervertebral disc along with two posterior facet joints cushion and dampen the various translational and rotational forces exerted upon the spinal column. The intervertebral disc is a spacer located between two vertebral bodies. The facets provide stability to the posterior portion of adjacent vertebrae. The spinal cord is housed in the canal of the vertebral bodies. It is protected posteriorly by the lamina. The lamina is a curved surface with three main protrusions. Two transverse processes extend laterally from the lamina, while the spinous process extends caudally and posteriorly. The vertebral bodies and lamina are connected by a bone bridge called the pedicle.

The spine is a flexible structure capable of a large range of motion. There are various disorders, diseases, and types of injury, which restrict the range of motion of the spine or interfere with important elements of the nervous system. The problems include, but are not limited to, scoliosis, kyphosis, excessive lordosis, spondylolisthesis, slipped or ruptured disc, degenerative disc disease, vertebral body fracture, and tumors. Persons suffering from any of the above conditions typically experience extreme and/or debilitating pain, and often times diminished nerve function. These conditions and their treatments can be further complicated if the patient is suffering from osteoporosis, or bone tissue thinning and loss of bone density.

There are many known spinal conditions, e.g., scoliosis, that require the imposition and/or maintenance of corrective forces on the spine in order to return the spine to its normal condition. As a result, numerous devices (e.g., alignment systems) have been developed for use in spinal fixation. One type of spinal construct may include, for example, one or more spinal rods that can be placed parallel to the spine with spinal fixation devices (such as hooks, screws, or plates) interconnected between the spinal rods at selected portions of the spine. The spinal rods can be connected to each other via cross-connecting members to provide a more rigid support and alignment system.

When a spinal rod is used as a support and stabilizing member, commonly, a series of two or more screws are inserted into two or more vertebrae to be instrumented. A spinal rod is then placed within or coupled to the heads of the screws, or is placed within a connecting device that links the spinal rod and the heads of the screws, and the connections are tightened. In this way, a rigid supporting structure is fixed to the vertebrae, with the spinal rod providing the support that maintains and/or promotes correction of the vertebral malformation or injury.

Flexibility during a surgical procedure in which a surgeon manipulates and positions the spinal vertebrae into desired alignment prior to locking the spinal rod into a rigid connection to the screws is important. Accordingly, a need exists for spinal fixation devices with improved insertion and fixation characteristics into bone that also provide flexibility during spinal column manipulation.

### SUMMARY

The present disclosure is directed to a spinal fixation device including a bone screw and a housing assembly to facilitate attachment of a spinal rod to the spinal fixation device in the treatment of spinal conditions that require manual realignment or positioning of the spinal column prior to locking the spinal fixation device and the spinal rod in a desired position.

In accordance with an aspect of the present disclosure, a spinal fixation device includes a housing assembly including an inner housing and an outer housing selectively slidable relative to each other between a locked position and an unlocked position, and a bone screw. The bone screw includes a head portion selectively securable within the inner housing of the housing assembly, a neck portion extending distally from the head portion, and an elongated body portion extending distally from the neck portion. The elongated body portion includes a proximal region and a distal region, the distal region including a threaded section having threads and an unthreaded section extending distally from the threaded section and terminating at a distal tip. The threaded section includes a cutting flute defined therein proximal to the unthreaded section.

The cutting flute may have a radius of curvature of about 13.7 mm to about 14.2 mm. In embodiments, the radius of curvature is 13.97 mm. A center of curvature of the cutting flute may be about 3.3 mm to about 3.8 mm from the distal tip. In embodiments, the center of curvature is about 3.56 mm from the distal tip.

A ratio of a length of the threaded section to a length of the unthreaded section may be about 3:1. A length of the unthreaded section of the distal region of the elongated body portion may be about 2.67 mm to about 2.92 mm. In embodiments, the length of the unthreaded section is about 2.79 mm. A length of the threaded section of the distal region of the elongated body portion may be about 8.94 mm to about 9.45 mm. In embodiments, the length of the threaded section is about 9.2 mm.

The threads of the threaded distal region may be dual lead threads. A pitch of the threads may be about 2.2 mm to about 2.4 mm. In embodiments, the pitch is about 2.25 mm. Each of the threads may have a leading angle of about 26° to about 28° and a trailing angle of about 2° to about 4°. In some embodiments, the leading angle is about 27° and/or the trailing angle is about 3°. The proximal region of the elongated body may include single lead threads.

The unthreaded section of the distal region of the elongated body may have a conical shape defining a cone angle of about 35° to about 37°. In embodiments, the cone angle is about 36°.

The neck portion of the bone screw may have a diameter that is at least equal to a major diameter of the elongated body portion.

Other aspects, features, and advantages will be apparent from the description, drawings, and the claims.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiments given below, serve to explain the principles of the disclosure, wherein:

FIG. 1 is a front view of a spinal fixation device in accordance with an embodiment of the present disclosure;

FIG. 2 is a side view of the spinal fixation device of FIG. 1;

FIG. 3 is a bottom perspective view of the spinal fixation device of FIGS. 1 and 2;

FIG. 4 is a top perspective view of the spinal fixation device of FIGS. 1-3;

FIG. 5 is a side view of a bone screw of the spinal fixation device of FIGS. 1-4;

FIG. 6 is a side cross-sectional view of the bone screw of FIG. 5, taken along line 6-6 of FIG. 5;

FIG. 7 is a close up view of the area of detail indicated in FIG. 6;

FIG. 8 is a perspective view of the bone screw of FIG. 5;

FIG. 9 is a side view of the bone screw of FIG. 5; and

FIG. 10 is a perspective view, with parts separated, of a spinal fixation system including the spinal fixation device of FIG. 1 and a spinal rod in accordance with an embodiment of the present disclosure.

### DETAILED DESCRIPTION

Embodiments of the present disclosure are now described in detail with reference to the drawings in which like reference numerals designate identical or corresponding elements in each of the several views. Throughout this description, the term “proximal” refers to a portion of a structure (e.g., a device or component thereof) closer to a user, while the term “distal” refers to a portion of the same structure further from the user. The terms “generally,” “substantially,” and “about” shall be understood as words of approximation that take into account relatively little to no variation in the modified term(s). Additionally, in the drawings and in the description that follows, terms such as “top,” “bottom,” “upward,” and “downward,” and similar directional terms are used simply for convenience of description and are not intended to limit the disclosure. In the following description, well-known functions or constructions are not described in detail to avoid obscuring the present disclosure in unnecessary detail.

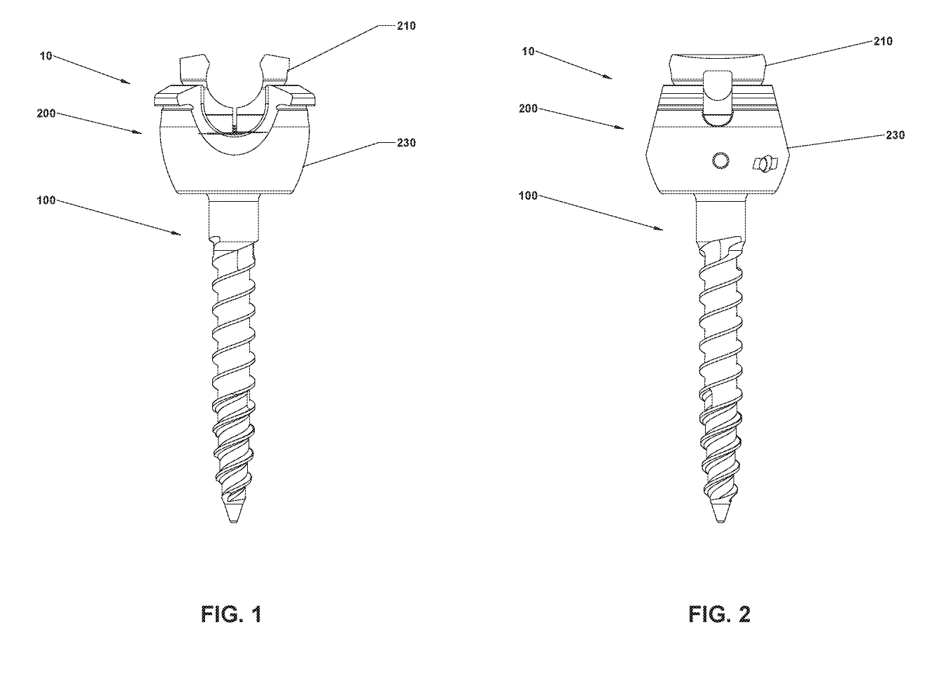

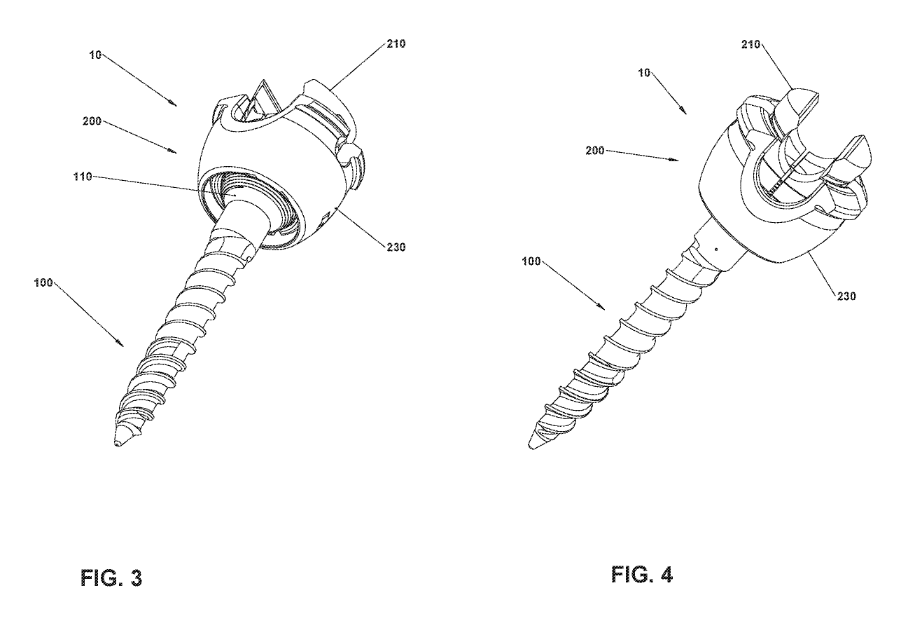

Referring now to FIGS. 1-4, a spinal fixation device 10 includes a bone screw 100 and a housing assembly 200 including an inner housing 210 and an outer housing 230. A head portion 110 (FIG. 3) of the bone screw 100 is configured for engagement with and movement relative to the inner housing 210 of the housing assembly 200, and the outer housing 230 of the housing assembly 200 is configured to receive at least a portion of the inner housing 210 therein.

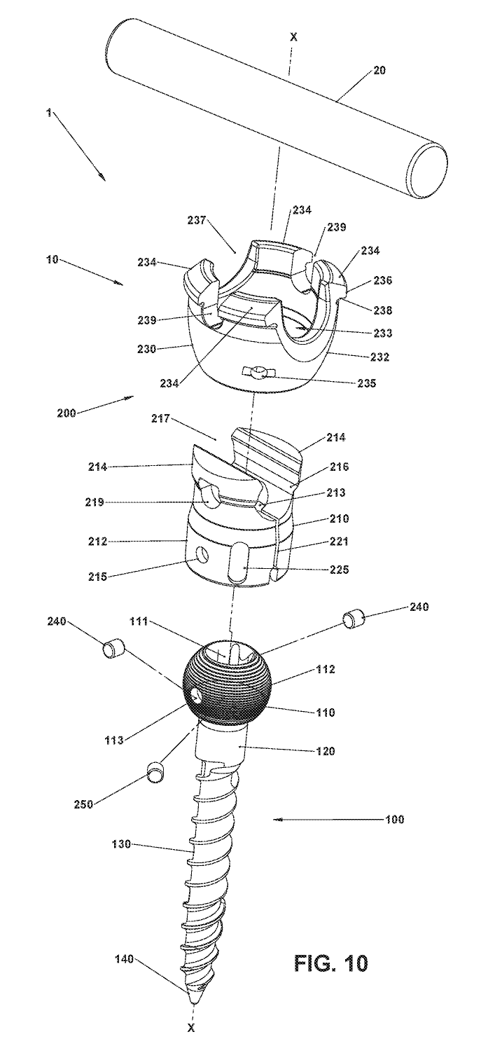

A taper lock is defined between the inner and outer housings 210, 220 such that in a first position, the spinal fixation device 10 is unlocked and able to receive a spinal rod 20 (FIG. 10) therein, and in a second position, the taper lock between the inner and outer housings 210, 220 is locked and compresses the inner housing 210 against the spinal rod 20 to secure the spinal fixation device 10 to the spinal rod 20.

The spinal fixation device 10 is formed from biocompatible material(s) including, but not limited to, metals, such as stainless steel, cobalt chrome, titanium, and titanium alloy, as well as various polymers (e.g., polyether ether ketone, polyphenylsulfone, polyetherimide, polycarbonate, polyethylene, polypropylene, polyacetal, or other such engineering resin), or combinations of the aforementioned materials.

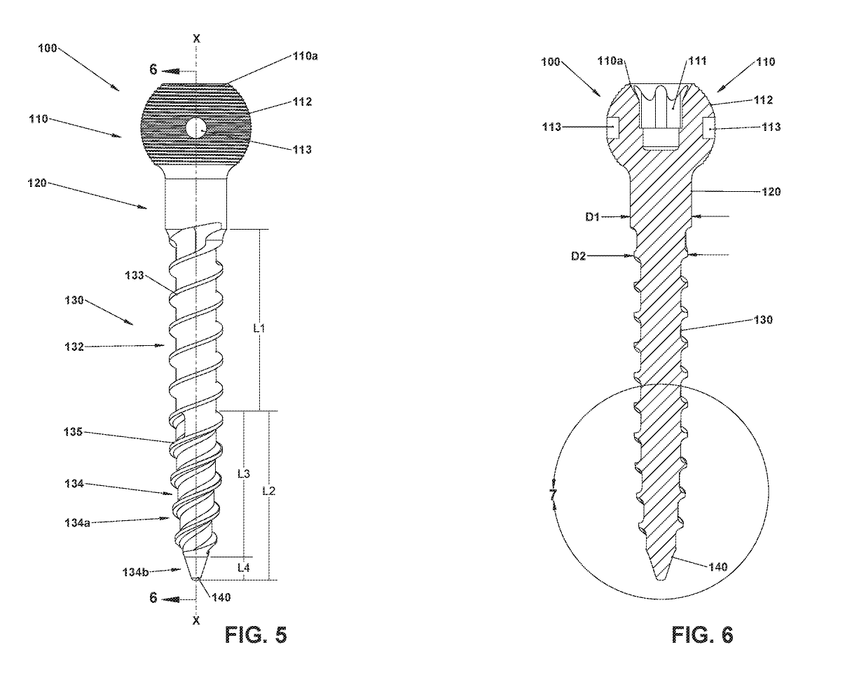

With reference now to FIGS. 5 and 6, the bone screw 100 extends along a longitudinal axis “X” and includes a head portion 110, a neck portion 120, and an elongated body portion 130 terminating at a distal tip 140. The head portion 110 is substantially spherical in shape and includes a textured outer surface 112 which serves to provide a degree of limited resistance to the operation of the spinal fixation device 10. Any type or degree of texturing or grooving of the outer surface 112 of the head portion 110 can be employed in the manufacture of the bone screw 100 so long as the articulation of the bone screw 100 relative to the housing assembly 200 (see e.g., FIG. 3) is not prohibited by the texturing/grooving.

A proximal end 110a of the head portion 110 includes a recess 111 defined therein that is configured to mate with a driving instrument (not shown). The recess 111 may have a hex feature, e.g., hexagonal or hexolobular in shape, or any other suitable configuration that is engageable and/or complementary with a suitable driving instrument to enable the driving instrument to control the insertion and/or advancement, as well as retraction and/or withdrawal, of the bone screw 100 into bone. Alternatively, the proximal end 110a of the head portion 110 can include a protrusion rather than a recess 111, provided that the protrusion has a surface that is configured for gripping attachment to a driving instrument and provided that the height of the protrusion above the proximal end 110a of the head portion 110 does not obstruct or interfere with any of the functions of the spinal fixation device 10.

The head portion 110 further includes cavities 113 defined in the outer surface 112 that are generally centered on the head portion 110 and positioned on directly opposing sides of the head portion 110. The cavities 113 are each configured to receive a pivot pin 240 (FIG. 10) to allow for pivotable movement of the head portion 110 relative to the inner housing 210 of the housing assembly 200. The cavities 113 may be a single cavity extending through the head portion 110 such that a single pivot pin is passed entirely therethrough and about which the head portion 110 is capable of pivoting in a single or uni-directional manner. Alternatively, the head portion 110 may not include the cavities 113.

The neck portion 120 of the bone screw 100 interconnects the head portion 110 and the elongated body portion 130. The neck portion 120 has a cylindrical shape and smooth outer surface. The diameter “D1” of the neck portion 120 is at least the same as, and in some embodiments, greater than a major diameter “D2” of the elongated body portion 130 such that during insertion of the bone screw 100 into bone, the opening formed in the bone is fully filled by the neck portion 120. Specifically, the neck portion 120 has an outer diameter that is substantially the same as the diameter of the opening formed in bone and this arrangement inhibits the introduction of foreign matter into the opening in bone. Additionally, it inhibits the bone screw 100 from pivoting within the opening in bone.

The elongated body portion 130 includes a first or proximal region 132 extending distally from the unthreaded neck portion 120 and a second or distal region 134 extending distally from the proximal region 132. The elongated body portion 130 has a helical thread pattern extending along a majority of the length thereof, with the proximal region 132 including single lead threads 133 which transition into dual lead threads 135 in the distal region 134. The threads 133 of the proximal region 132 extend uniformly along the entire length “L1” of the proximal region 132, and the threads 135 of the distal region 134 extend along the entire length “L3” of a threaded section 134a of the distal region 134, terminating proximal to an unthreaded section 134b of the distal region 134.

The length “L1” of the proximal region 132 may be about 15.3 mm to about 75.4 mm. It should be understood that the length “L1” of the proximal region 132 may depend on, for example, the insertion depth and/or orientation of the bone screw 100 into bone, among other factors as within the purview of those skilled in the art. The length “L2” of the distal region 134 may be about 11.75 mm to about 12.25 mm. In embodiments, the length “L2” of the distal region 134 is about 11.87 mm to about 12.12 mm, and in some embodiments, the length “L2” is about 11.99 mm. The length “L3” of the threaded section 134a of the distal region 134 may be about 8.94 mm to about 9.45 mm. In embodiments, the length “L3” of the threaded section 134a is about 9.07 mm to about 9.32 mm, and in some embodiments, the length “L3” is about 9.2 mm. The length “L4” of the unthreaded section 134b of the distal region 134 may be about 2.67 mm to about 2.92 mm. In embodiments, the length “L4” of the unthreaded section 134b is about 2.73 mm to about 2.86 mm, and in some embodiments, the length “L4” is about 2.79 mm. The ratio of the lengths “L3,” “L4” of the threaded and unthreaded sections 134a, 134b may be about 3:1.

The proximal region 132 has a non-tapering profile along the length “L1” thereof and may have a major diameter “D2” of about 3.89 mm to about 6.6 mm. In embodiments, the major diameter “D2” is about 4.28 mm to about 5.64 mm, and in some embodiments, the major diameter “D2” is about 4.67 mm. The distal region 134 has a tapering profile that tapers distally towards the distal tip 140. The distal region 134 may aid in driving the bone screw 100 into bone, and the proximal region 132 of the elongated body 130 may enhance anchoring of the bone screw 100 in bone.

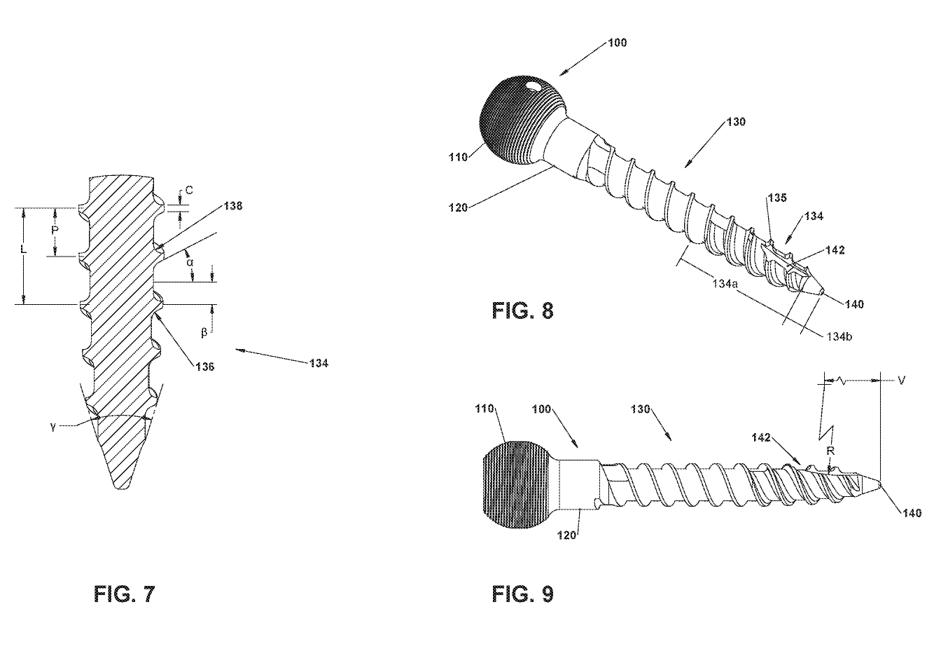

As shown in FIG. 7, the pitch “P” of the threads 135 in the distal region 134 may be about 2.2 mm to about 2.4 mm. In embodiments, the pitch “P” is about 2.03 mm to about 2.33 mm, and in some embodiments, the pitch “P” is about 2.25 mm. In embodiments, the pitch of the threads 133 (FIG. 5) in the proximal region 132 of the elongated body 130 is substantially the same as the pitch “P” of the threads 135 in the distal region 134, with the lead “L” of the distal region 134 being two times the pitch “P”.

Each thread 135 of the distal region 134 has a leading edge 136 that extends at a leading angle “α”. The leading angle “α” may be about 26° to about 28°. In embodiments, the leading angle “α” is about 26.5° to about 27.5°, and in some embodiments, the leading angle “α” is about 27°. Each thread 135 also has a trailing edge 138 that extends at a trail angle “β”. The trailing angle “β” may be about 2° to about 4°. In embodiments, the trailing angle “β” is about 2.5° to about 3.5°, and in some embodiments, the trailing angle “β” about 3°. A crest “C” of each thread 135 may have a width of about 0.2 mm to about 0.41 mm. In embodiments, the crest “C” is about 0.25 mm to about 0.35 mm, and in some embodiments, the width of the crest “C” is about 0.3 mm. The configuration of the threads 135 of the distal region 134 enhances the pullout resistance properties of the bone screw 100.

The unthreaded section 134b of the distal region 134 has a conical shape terminating at the distal tip 140. The distal tip 140 may have a blunted or pointed profile, among other configurations suitable for engaging and/or piercing bone into which the bone screw 100 is to be placed. The unthreaded section 134b defines a cone angle “γ” which may be about 35° to about 37°. In embodiments, the cone angle “γ” is about 35.5° to about 36.5°, and in some embodiments, the cone angle “γ” is about 36°.

As shown in FIGS. 8 and 9, a cutting flute 142 is defined in the distal region 134 of the elongated body portion 130 in the threaded section 134a proximal to the unthreaded section 134b (i.e., the cutting flute 142 terminates prior to and does not extend into the unthreaded region 134b). The radius of curvature “R” of the cutting flute 142 may be about 13.7 mm to about 14.2 mm. In embodiments, the radius of curvature “R” is about 13.84 mm to about 14.08 mm, and in some embodiments, the radius of curvature is about 13.97 mm. The center of curvature “V” of the cutting flute 142 may be about 3.3 mm to about 3.8 mm from the distal tip 140 to the center of the cutting flute 142. In embodiments, the center of curvature “V” is about 3.43 mm to about 3.68 mm from the distal tip 140, and in some embodiments, the center of curvature “V” is about 3.56 mm from the distal tip 140.

The configuration of the distal region 134 of the elongated body portion 130 including, for example, the cone angle “γ”, the position of the cutting flute 142, the configuration of the threads 135, and/or the shape of the unthreaded section 134b and the distal tip 140, facilitates locating and/or inserting of the distal tip 140 into a pre-drilled hole in bone, while minimizing or preventing the bone screw 100 from cutting a path away from the pre-drilled hole.

With reference now to FIG. 10, the inner housing 210 of the housing assembly 200 has a generally cylindrical body portion 212 including an opening 213 extending axially therethrough. The opening 213 is sized to retain the head portion 110 of the bone screw 100 therein. An inner surface (not shown) of the inner housing 210 defining the opening 213 has a complementary surface configuration to the head portion 110 of the bone screw 100 so as to facilitate articulation of the head portion 110 within the opening 213. Opposed through holes 215 are defined in the body portion 212 of the inner housing 210 and aligned with the cavities 113 of the head portion 110 of the bone screw 100. The through holes 215 and the cavities 113 are configured to receive the pivot pins 240 such that the head portion 110 is capable of pivoting thereabout in a single or uni-directional manner relative to the inner housing 210. Alternatively, if a greater range of motion is desired, the pivot pins 240 may be omitted such that the head portion 110 is capable of pivoting in a multi-axial or polyaxial manner relative to the inner housing 210.

Upstanding wings 214 extend from the body portion 212 and define a saddle 217 having a generally U-shaped configuration. The saddle 217 is configured and dimensioned for receiving at least a portion of a spinal rod 20 transversely therein. Each wing 214 includes at least one contact surface 216 that when forced into compressive contact with the spinal rod 20 present in the saddle 217, serves to securely hold the spinal rod 20 in its relative position to the inner housing 210. Each wing 214 further includes a recess 219 defined in an outer surface thereof that is engageable with a suitable grasping instrument (not shown) to enable the grasping instrument to unlock the inner and outer housing 210, 230 relative to each other.

The body portion 212 further includes a slot 221 and a guide pin slot 225 defined therethrough. The slot 221 extends through the body portion 212 to allow the body portion 212 to flex inward and outward in response to compressive and tensile forces applied to the body portion 212, and to permit the wings 214 to flex towards and away from each other to vary the dimension of the saddle 217. Allowing the saddle 217 to vary in size permits the inner housing 210 to accommodate spinal rods having differing diameters and/or shapes. Although a full slot 221 is shown, it should be understood that the slot 221 may extend only partially through the body portion 212 (e.g., does not extend through the bottom of the body portion 212). The guide pin slot 225 is configured to slidably receive a guide pin 250 therein such that the inner housing 210 is axially movable with respect to the outer housing 230.

The outer housing 230 includes an annular body portion 232 having an opening 233 extending axially therethrough. The opening 233 is configured to receive at least a portion of the inner housing 210 therein. The outer housing 230 includes a guide pin opening 235 that is aligned with the guide pin slot 225 of the inner housing 210 such that the outer housing 230 is capable of selectively sliding over a portion of the inner housing 210 in an upward and downward direction along the longitudinal axis “X” of the bone screw 100. The sliding movement of the outer housing 230 relative to the inner housing 210 is facilitated by the guide pin 250 which is sized and configured for insertion through the guide pin opening 235 defined in the outer housing 230 and sliding movement within the guide pin slot 225 of the inner housing 210.

The outer housing 230 includes a plurality of fingers 234 extending from the body portion 232 that define a saddle 237 having a generally U-shaped configuration and slots 239 through which the recesses 219 of the wings 214 of the inner housing 210 may be accessed. The saddle 237 is configured and dimensioned for receiving the spinal rod 20. The configuration of both the inner and outer housings 210, 230 are complementary such that when the outer housing 230 is slid upward in relation to the inner housing 210 to align the plurality of fingers 234 of the outer housing 230 with the wings 214 of the inner housing 210, the plurality of fingers 234 compress the wings 214 against the spinal rod 20 to secure the spinal rod 20 to the spinal fixation device 10.

Each finger 234 includes an annular flange 236 that extends radially from the finger 234 and a gripping groove 238 located beneath the annular flange 236. Together, the annular flange 236 and the groove 238 are configured to receive a grasping tool (not shown) to move the outer housing 230 relative to the inner housing 210.

Prior to use of the spinal fixation device 10, the housing assembly 200 should be in the unlocked, first position with the outer housing 230 slid downward relative to the inner housing 210 (see e.g., FIG. 1). In a method of use, the elongated body portion 130 of the bone screw 100 is driven into bone by providing torsional force via a driving instrument (not shown) configured to mate with the recess 111 defined in the proximal end 110a of the head portion 110 of the bone screw 100. Typically, the bone screw 100 will be inserted into a pre-drilled hole in bone. After the elongated body portion 130 and the neck portion 120 of the bone screw 100 are positioned within the hole and the driving instrument is removed from the bone screw 100, the spinal rod 20 is positioned transversely along the common course of and within the saddles 217, 237 of the inner and outer housings 210, 230 of the housing assembly 200. The configuration of the spinal fixation device 10 allows for mono-axial or poly-axial rotational movement of the housing assembly 200 relative to the bone screw 100. This articulation facilitates positioning of the spinal rod 20 relative to the bone screw 100 and allows the user (e.g., a surgeon) to manipulate the spinal column as needed.

Following completion of all spinal manipulation, the spinal fixation device 10 is locked to the spinal rod 20 in the selected/desired position. To achieve locking of the spinal fixation device 10, the outer housing 230 is grasped by the user using a grasping tool (not shown) that slides the outer housing 230 upwards and over the inner housing 210 from the unlocked, first position to the locked, second position. Additionally, the user may use a grasping instrument (not shown) to grasp the inner housing 210 and slidably move the outer housing 230 downwards along the inner housing 210 to unlock the spinal fixation device 10.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown and described in connection with certain embodiments may be combined with the elements and features of certain other embodiments without departing from the scope of the present disclosure, and that such modifications and variation are also included within the scope of the present disclosure. Accordingly, the subject matter of the present disclosure is not limited by what has been particularly shown and described.

What is claimed is:

1. A spinal fixation device comprising:
a housing assembly including an inner housing and an outer housing selectively slidable relative to each other between a locked position and an unlocked position; and
a bone screw including:
a head portion selectively securable within the inner housing of the housing assembly;
a neck portion extending distally from the head portion; and
an elongated body portion extending distally from the neck portion, the elongated body portion including a proximal region and a distal region, the distal region including a threaded section having threads and an unthreaded section extending distally from the threaded section and terminating at a distal tip, the threaded section including a cutting flute defined therein proximal to the unthreaded section.

2. The spinal fixation device of claim 1, wherein the cutting flute has a radius of curvature of about 13.7 mm to about 14.2 mm.

3. The spinal fixation device of claim 2, wherein the radius of curvature is 13.97 mm.

4. The spinal fixation device of claim 2, wherein a center of curvature of the cutting flute is about 3.3 mm to about 3.8 mm from the distal tip.

5. The spinal fixation device of claim 4, wherein the center of curvature is about 3.56 mm from the distal tip.

6. The spinal fixation device of claim 1, wherein a ratio of a length of the threaded section to a length of the unthreaded section is about 3:1.

7. The spinal fixation device of claim 1, wherein a length of the unthreaded section of the distal region of the elongated body portion is about 2.67 mm to about 2.92 mm.

8. The spinal fixation device of claim 7, wherein the length of the unthreaded section is about 2.79 mm.

9. The spinal fixation device of claim 7, wherein a length of the threaded section of the distal region of the elongated body portion is about 8.94 mm to about 9.45 mm.

10. The spinal fixation device of claim 9, wherein the length of the threaded section is about 9.2 mm.

11. The spinal fixation device of claim 1, wherein the threads of the threaded distal region are dual lead threads.

12. The spinal fixation device of claim 11, wherein a pitch of the threads is about 2.2 mm to about 2.4 mm.

13. The spinal fixation device of claim 12, wherein the pitch is about 2.25 mm.

14. The spinal fixation device of claim 11, wherein each of the threads has a leading angle of about 26° to about 28° and a trailing angle of about 2° to about 4°.

15. The spinal fixation device of claim 14, wherein the leading angle is about 27°.

16. The spinal fixation device of claim 14, wherein the trailing angle is about 3°.

17. The spinal fixation device of claim 11, wherein the proximal region of the elongated body includes single lead threads.

18. The spinal fixation device of claim 7, wherein the unthreaded section of the distal region of the elongated body has a conical shape defining a cone angle of about 35° to about 37°.

19. The spinal fixation device of claim 18, wherein the cone angle is about 36°.

20. The spinal fixation device of claim 1, wherein the neck portion has a diameter that is at least equal to a major diameter of the elongated body portion.
